(19) United States
(12) Patent Application Publication
(10) Pub. No.: US 2003/0175853 A1
(43) Pub. Date: Sep. 18, 2003

(54) Delayed release of fluids

(76) Inventors: David J Clarke, Cheshire (GB); Andrew Smith, Cheshire (GB)

Correspondence Address: NIXON & VANDERHYE, PC, 1100 N GLEBE ROAD, 8TH FLOOR, ARLINGTON, VA, 22201-4714, US

(21) Appl. No.: 10/182,187

(22) PCT Filed: Jan. 25, 2001
(86) PCT No.: PCT/GB01/00274

(30) Foreign Application Priority Data
Jan. 25, 2000 (GB) 0001682.4
Jan. 25, 2000 (GB) 0001683.4

(51) Int. Cl. C12P 1/00

(52) U.S. Cl. 435/41

(57) ABSTRACT

A method of effecting delayed release of a fluid (19) comprises providing the fluid in a vessel (12) having a discharge aperture (14) provided with a polymeric barrier (5) to fluid release. The barrier (15) is caused to degrade to provide for failure of the barrier and discharge of the fluid. The liquid to be discharged may be an aqueous medium and the barrier (15) may be of a polymeric material capable of forming in the presence of the aqueous medium a gel that will dissolve therein so as to cause failure of the barrier. The polymeric material may be a cellulose ether. The fluid may, for example, comprise a culture of microorganisms which are subjected to pre-enrichment in the vessel for a predetermined period of time prior to failure of the barrier (15) and discharge of the culture into, for example, a selective medium.

The present invention relates to a method for the delayed release of fluids and to a delayed release device capable of holding a fluid and discharging that fluid after a certain period of time. The method and device may for example be for use in treating a culture of microbial organisms and discharging the treated culture, e.g. for the purposes of a subsequent operation to be effected on the culture. The invention is useful particularly, but by no means exclusively, to the isolation of microorganisms, e.g. for the purpose of analysing food samples for the presence of Salmonella or other bacteria.

There are well established procedures for detecting Salmonella and other bacteria. Typically, the procedure for detecting Salmonella in an environmental sample (e.g. food) involves an initial pre-enrichment step and subsequent step of selective enrichment. In the first of these steps, the sample under test is incubated in a medium which encourages micro-organism recovery and growth. The pre-enrichment medium may for example be buffered peptone water. For the second step (selective enrichment) an aliquot of the cultured pre-enrichment medium is transferred into a selective medium (e.g. Rappaport Vassiliadis enrichment broth) allowing selective growth of the target micro-organisms to detectable levels.

A development of the above technique is disclosed in WO-A-9902650 (Oxoid Ltd.) in which a powdered selective medium is contained within a delayed release capsule provided in the pre-enrichment medium, said capsule allowing for release of the selective medium into the pre-enrichment culture after a certain period of time. More particularly, the capsules for use in the technique disclosed in WO-A-9902650 comprise a capsule body containing the powdered selective medium and having an opening in which is located a water swellable hydrogel plug so as initially to retain the selective medium in the capsule. With the capsule located in the pre-enrichment medium, the hydrogel plug is exposed to aqueous media and during the pre-enrichment culturing step swells until it disengages from the neck of the capsule body to allow the selective medium to be released “automatically” into the pre-enrichment medium so that the step of selective growth of the target micro-organisms may be effected.

There are however a number of disadvantages associated with the use of the delayed release capsule disclosed in WO-A-9902650. Firstly, there is the drawback of the amount of selective medium which can be incorporated into the capsule (without making the capsule unduly large) as witnessed by the fact that six of the capsules are provided to deliver the selective medium into 225 ml of pre-enrichment medium. Secondly, when the plugs are disengaged from the capsule, mixing of the selective medium with the pre-enrichment medium is by diffusion and will be relatively slow. Thirdly, with the plugs of the type employed in WO-A-9902650 it is difficult accurately to deliver the active agents over long release periods.

WO-A-9926853 discloses a delayed release device for delivering a liquid (usually an aqueous liquid) after a predetermined time delay. The device of WO-A-9926853 comprises a container (within which the liquid to be delivered is contained) having an outlet closed by a swellable plug of the type disclosed in WO-A-9902650.In use, the device is positioned such that the plug is lowermost so that the contents of the container are in contact with the plug to allow swelling thereof. After a predetermined period of time, the plug becomes disengaged from the outlet and the contents of the container are discharged under gravity.

According to a first aspect of the present invention there is provided a method of effecting delayed release of a fluid, the method comprising providing the fluid in a vessel having a discharge aperture provided with a polymeric barrier to fluid release, and causing the barrier to degrade to provide for failure of the barrier and discharge of the fluid.

The degradation of the barrier may occur after a predetermined period of time for which the fluid has been in contact with the barrier which therefore provides for delayed release of the contents of the vessel.

The fluid to be discharged may be a gas, vapour or more preferably a liquid. If the fluid to be discharged is a liquid then it may be an aqueous or organic liquid.

Thus, in the method of the invention, release of the fluid is as a result of degredation of the barrier after a period of time (providing for the delayed release). The method of the invention is to be distinguished from WO-A-9902650 in which the polymeric plug swells to such a size that it becomes dislodged from the opening in which it was originally located whereas, in the method of the invention, the barrier is degraded to allow liquid to be discharged. This discharge may occur as a result of loss of mechanical integrity of the barrier such that it is no longer able to withstand the force of the liquid against it.

The polymeric barrier may for example be in the form of a disc having parallel faces. Alternatively, one of the faces of the discs may be either convex or concave and it may this be this face or the planar face which is in contact with the liquid. By adjusting the shape of the polymeric barrier in this way it is possible to provide for different release times. In other words, the release time is a function of the shape of the barrier.

The polymeric barrier may, for example, be a close fit in the discharge aperture and failure of the barrier may result as a result of dissolution or the degradation of the barrier around its edges so that it is no longer able to withstand the force of liquid.

In a further embodiment, the barrier may be provided in the form of a cartridge comprised for example of the polymeric barrier material per se (e.g. in the form of a disk) and a “frame” provided around the perimeter of polymeric material. Such cartridges are easy to handle and may be located on a “seat” in the apparatus so as to be readily replaced as necessary.

Alternatively, the polymeric barrier may be supported over the discharge aperture by a mesh or the like serving to locate the barrier in position but to allow passage of liquid on failure of the barrier.

It is particularly preferred, in accordance with the invention, that it is fluid provided in the vessel which causes degradation of the barrier to provide for failure thereof. The degradation of the barrier is for preference caused by the fluid to be discharged. An alternative possibility is to add to the vessel a further fluid (in addition to that which is the subject of the delayed release) and it is this further fluid which effects degradation of the barrier. In all instances, it is preferred that the fluid which effects degradation of the barrier is a liquid, preferably an aqueous liquid but possibly also an organic liquid.

Thus, for example, the barrier may undergo at least partial dissolution by the fluid to provide for barrier failure and fluid discharge.

Alternatively, the fluid may convert the barrier to a gel and the gel at least partially dissolves in the fluid to provide for failure of the barrier.

A further possibility is that the barrier may be provided on the form of a gel and fluid in the vessel desolvates the gel resulting in barrier failure.

A still further possibility is that the barrier is of at lease partially crystalline material (the crystallinity serving to provide the integrity of the barrier) and the fluid disrupts the crystallinity to provide for barrier failure.

In an alternative embodiment of the invention failure of the barrier may be effected by energy provided from externally of the vessel. For example, degradation of the barrier by a laser beam generated externally of the vessel is one possibility. A further possibility is the use of ultra-sound. Such external means of effecting barrier failure may be sole means of instigating barrier failure or may be used to enhance the effect of failure caused by fluid within the vessel (which provides barrier failure by one of the abovedescribed mechanisms)

The method of the invention is particularly suitable for use in treating a culture of microbial organisms and effecting discharge of the treated culture.

Thus according to a second aspect of the present invention there is provided a method of treating a culture of microbial organisms in a liquid medium comprising providing the liquid medium (containing the microbial organisms) in a vessel having a discharge aperture provided with a polymeric barrier to liquid flow, effecting the treatment, and utilising liquid in the vessel to cause the barrier to degrade to undergo a loss of mechanical strength to integrity to provide for failure of the barrier and discharge of the liquid medium.

In accordance with a further aspect, the invention provides apparatus for use in the method of the invention comprised of a vessel capable of holding a liquid medium (e.g. a culture of microbial organisms in a liquid medium) and having a discharge aperture provided with a polymeric barrier to liquid release, said barrier being capable of undergoing degradation in the vessel to provide for failure of the barrier and discharge of liquid medium wherein said degradation is effected by the liquid.

The treatment effected to the culture may, for example, be recovery, resuscitation, growth or enrichment of the microbial organisms. The treatment may include sustained release of an agent into the culture. This agent may, for example, be a carbon source to encourage growth or a selective agent.

The degradation of the barrier may occur after a predetermined period of time for which the liquid has been in contact with the barrier which therefore provides for delayed release of the contents of the vessel.

Thus the method of the second aspect of the invention allows a culture of a microbial organism to be treated over a period of time in a vessel and then to be released from the vessel (e.g. for the purpose of a subsequent operation to be effected on the culture) by virtue of liquid in the vessel causing failure of a polymeric barrier originally closing a liquid outlet of the vessel.

The liquid which causes the barrier to degrade may be an aqueous medium or an organic medium.

The barrier may undergo at least partial dissolution by the liquid to provide for barrier failure and liquid discharge. Alternatively, the liquid may convert the barrier to a gel and the gel at least partially dissolves in the liquid to provide for failure of the barrier.

The method and device of the invention are particularly suitable for use in isolating micro-organisms for the purposes of detection. The device preferably comprises the vessel as a first chamber and a second chamber connected to the first chamber via said discharge aperture in which the polymeric barrier is provided. Preferably the second chamber is external of the first chamber. Preferably also the first chamber is an upper chamber and the second chamber is a lower chamber.

For the purpose of carrying out a test to determine the presence or otherwise of a micro-organism in a sample (e.g. of food), the sample and an aqueous pre-enrichment medium are provided in the upper chamber and a selective medium (which may be a powder or liquid) is provided in the lower chamber. The polymeric barrier in this embodiment is preferably one which, in the presence of the aqueous medium, forms a gel which dissolves in the medium. As the pre-enrichment step proceeds in the upper chamber, the barrier (by virtue of its contact with the aqueous medium) is converted into a gel which dissolves into the aqueous pre-enrichment medium. At a particular time (as determined by the composition and thickness of the barrier) a point is reached at which the barrier cannot withstand the pressure the media applies to it and the barrier fails releasing the pre-enrichment media into the selective media. Thus the selective enrichment phase may proceed so as to result in isolation of the microorganism of interest and growth to detectable levels.

The procedure as described in the preceding paragraph has a number of advantages over that disclosed in WO-A-9902650.In particular, mixing of all of the pre-enrichment culture and the selective medium occurs relatively quickly since the former is “dropped” into the latter (as compared to the mixing by diffusion in WO-A-9902650). This overcomes a disadvantage associated with the use of a plurality of separate capsules of the selective medium in the pre-enrichment medium where the contents of the capsules might be released at different times. A further advantage is that relatively long delayed release times (e.g. up to 24 hours) may be provided where required.

In a further embodiment of the device, the lower chamber is sub-divided into a plurality of compartments each of which may contain a different selective medium for the selective enrichment of different pathogens (or different media for enrichment of the same pathogen), the device being such that portions of the pre-enrichment culture from the upper chamber are received in the individual compartments of the second chamber. This may be achieved in a number of ways. Thus, for example, the upper chamber may have a single discharge aperture positioned such that pre-enrichment culture is discharged into all of the individual compartments. Alternatively, the upper chamber may have a plurality of discharge apertures formed as a grid (each provided with a gellable barrier as described) positioned one above each of the compartments of the lower chamber.

As an alternative to pre-enrichment in the upper chamber (which involves both resuscitation and growth) it is possible for only the step of resuscitation to be carried out in the upper chamber.

Although it is preferred that the first and second chambers are upper and lower chambers respectively, other configurations for the device are possible. Thus, for example, the device may be such that the first and second chambers are horizontally (rather than vertically) disposed relative to each other.

The device may be used, for example, for the isolation of Salmonella for which purpose the pre-enrichment medium may be buffered peptone water (BPW), although re-formulated (as compared to that used in the conventional method) as a resuscitation medium. In the conventional method, an aliquot of the pre-enrichment culture (in BPW) is transferred to the selective medium (RV) and there must be outgrowth of Salmonella (if present) in the pre-enrichment medium to ensure the Salmonella are transferred in the aliquot. In the present invention, all of the media in the upper chamber is transferred so that the requirement for outgrowth is avoided and only recovery of the bacteria is required. As such, BPW may be reformulated so that the meat peptone concentration is reduced from the conventionally used value of 10 gl⁻¹ to 0.1 gl⁻¹. A reduction in the nutritional source in the pre-enrichment media will reduce the overall bacterial growth in this media and the number of organisms transferred (in the whole of the pre-enrichment media) will be similar to the number present in the aliquot of the conventional method. Consequently, the present invention allows a reduction in the length of the pre-enrichment media and/or increase in the sensitivity of the media.

A further advantage of reducing the nutritional content of the pre-enrichment media is that fewer competitors are transferred to the selective enrichment media so that the enrichment of Salmonella is not comprised. This overcomes the problem known as “protective crowding effect” whereby competitors transferred over from the pre-enrichment phase adversely affect enrichment of Salmonella.

Rappaport Vassiliadis employed as selective enrichment broth may be reformulated to reduce the ionic (salt) concentration as more salt will be transferred from the pre-enrichment media than would occur in the conventional regime.

Furthermore, an acid should be included in the RV medium to ensure that when the pre-enrichment medium (buffered pH=7.2) is added thereto, the resultant pH is 5.2 as required by the selective enrichment media. The acid may, for example, be an inorganic acid, e.g. HCl, (although the selective enrichment components must then exist as a concentrated liquid) or an organic acid, e.g. maleic acid, citric acid, succinic acid, or potassium hydrogen phthalate. Since these organic acids exist as powders, the reformulated RV media may be a powder.

If desired, provision may be made for sustained release into the pre-enrichment/resuscitation medium of an agent influencing growth of Salmonella. The agent may for example be glucose, meat peptone or any other carbon source to encourage growth. Alternatively there may be sustained release of a selective agent. The sustained release may be achieved, for example, by incorporating the agent in a sustained release tablet (e.g. of hydroxypropyl methylcellulose) added to the pre-enrichment/resuscitation medium. Alternatively, the agent may be incorporated in the barrier during manufacture thereof so that the agent is released from the barrier into the pre-enrichment/resuscitation medium. In a preferred embodiment, the barrier is formed of or incorporates “intelligent-type” polymer providing for release of the agent at a particular stage of bacterial growth. A review of such polymers is given in Macromol Symp. 98, 645-664 (1995).

It will of course be appreciated that the device may be applied to the isolation of microorganisms other than Salmonella, e.g. Listeria, E-Coli, Staphylococci and Campylobacter.

The above described procedures for isolating a microorganism of interest will generally be carried out at a temperature of 35° to 45° C. (typically about 37° C.) but we do not preclude values outside this range. Thus, lower or higher temperatures may be used for the enrichment of Psychrophilers or Thermophilers respectively.

The barrier may be adapted in various ways. Thus, for example, the possibility of incorporating an agent (e.g. glucose) for sustained release has been mentioned above. A further possibility is for the barrier to be coated (at least on its surface(s) exposed to the pre-enrichment media) with a pH responsive polymer which dissolves to expose the gellable polymeric material to the pre-enrichment media when the latter has reached a pre-determined pH (representative of a particular degree of pre-enrichment). Thus it is only when a particular degree of pre-enrichment has been attained that the barrier may begin to form a gel which then dissolves to provide for failure of the barrier.

It will be appreciated that the discharge outlet may be provided with more than one type of barrier. Thus, for example. There may be two barriers, one containing an agent for sustained release and the other not. Alternatively there may be two barriers each containing a different agent for sustained release, the agent from the lowermost barrier only beginning to be released on failure of the uppermost barrier. This arrangement allows the growth dynamics of the pre-enrichment to be manipulated. It is also possible to include more than two barriers incorporating agents for sustained release. A further possibility is to use, in conjunction with the two or more barriers containing agents for sustained release, a barrier which does not contain such an agent.

For all of the above arrangements, it is possible for at least one of the barriers to have a coating of a pH responsive polymer as described above.

Although the device of the invention has particular utility for detecting microorganisms, other uses may be envisaged. Thus, for example, the device could be adapted for use as a delayed release drip feed in medical applications.

Whilst for convenience the device of the invention uses gravity to transfer liquid from the first chamber to the second chamber on failure of the barrier other methods of transferring the liquid may be envisaged. Thus, for example, the device may comprise a capillary structure and utilise electroosmotic force to effect liquid movement on failure of the barrier. Alternatively, liquid may move under capillary action or under pressure of a gas. A further possibility is the use of a vacuum.

It is particularly preferred in accordance with the invention that the liquid medium to be transferred through the discharge aperture is an aqueous medium and that the barrier is of a polymeric material capable of forming, in the presence of the aqueous medium, a hydrated gel which will dissolve in the medium to provide for failure of the barrier and discharge of the liquid through said aperture.

Suitable polymers for use in forming such barriers are cellulose ethers containing methyl and preferably also hydroxypropyl groups. Further examples of cellulose which may be used include hydroxymethyl cellulose, hydroxyethyl cellulose and hydroxypropyl cellulose. Preferred ethers contain 15% to 35% substitution by methoxyl groups optionally (but preferably) in conjunction with 4% to 15% substitution by hydroxypropyl groups. A particularly preferred polymer for use in forming the barrier is one containing 19 to 24% methoxyl substitution and 7 to 12% hydroxypropyl substitution.

Particularly preferred are cellulose ethers of the type defined having a viscosity of 50 to 100 000 CP for a 2% solution.

Suitable polymers are available from Colorcon (Dow Chemical Company) under the trade mark METHOCEL and are available as grades A, E, F and K, each of which is available in a range of molecular weights. A particularly suitable polymer is available under the designation METHOCEL K100LV.

The physical diversity and gelation abilities of the METHOCEL range of polymers makes them suitable candidates as polymer barriers for use in the invention because the thickness of the barrier will dictate the delay in release. Furthermore, they are pH stable and their cellulose based structure is unlikely to be metabolised or have any bacteriostatic affect on bacterial cultures.

Additional polymers which may be cited for use in forming the barrier are available under the Trademarks POLOXMER and EUDRAGIT

Barriers formed from polymer as described above may be produced from the powdered polymeric material by the use of tableting equipment widely employed in the pharmaceutical industry. The force required for plug formation will be dependent on the formulation of the barrier and the size and shape thereof. Thus, for example, using the METHOCEL polymers described above, barriers may be prepared on a Beckman single punch press using a die and punch of 19 mm diameter and a force of 4 metric tonnes (equating to a pressure of 42.7 kg mm⁻²) for a period of 30 seconds.

The time for which the barrier is able to maintain sufficient integrity to prevent discharge of the aqueous medium (i.e. the delayed release time) will depend on the thickness of the barrier and also on factors such as the polymeric material from which the barrier is produced, and the nature and amount of the aqueous medium. To produce a barrier giving the required delayed release time for any particular application, it is a simple matter for the person skilled in the art to test a range of barriers of different thicknesses and/or of different polymeric materials to identify the barrier having the required delayed release time.

We have found for many applications that barriers produced by compression of METHOCEL L100KV under a pressure of 4 metric tonnes and having thicknesses of 0.6 mm to 1.6 mm will provide for delayed release times of about 1 to 18 hours when in contact with deionised water.

Although it is preferred that the barrier is of a material which, in the presence of an aqueous medium, will form a gel which dissolves in the medium to provide for failure of the barrier there are other possibilities. Thus, for example, the barrier may be formed of a hydrated gel which is stable only in the presence of water. By providing an alcohol in the liquid medium, the gel will become at least partially dehydrated to provide for barrier failure and release of the liquid medium.

A further possibility is that the liquid to be released is an organic liquid and the barrier is of a polymeric material which is either dissolved by the liquid or forms, in contact therewith, a gel which dissolves in the liquid to provide for barrier failure. The use of barriers which are degraded (to provide for barrier failure) may be used for concentration of bacteria or other microbial species by phase partitioning. Thus, for example, the device of the invention may be provided with barrier of a polymeric material degradable by oil and there is introduced into the device layer of oil in contact with the barrier. To the oil is added an aqueous medium containing bacteria which concentrate by partitioning at the interface of the aqueous and oil phases. Subsequently, the barrier fails by virtue of its contact with the oil medium which may then pass through the discharge aperture. Means may be provided for ensuring that only the oil medium (or that medium and a small proportion of the aqueous medium) is transferred through the discharge aperture whereby a more concentrated sample of the bacteria is obtained.

It will be appreciated that the polymeric materials may be formulated in various ways for example, mixtures of polymers may be used to provide the required delayed release time. Furthermore, the barrier may include fillers to provide desired properties for the barrier.

The invention will be further described, by way of example only, with reference to the accompanying drawings in which:

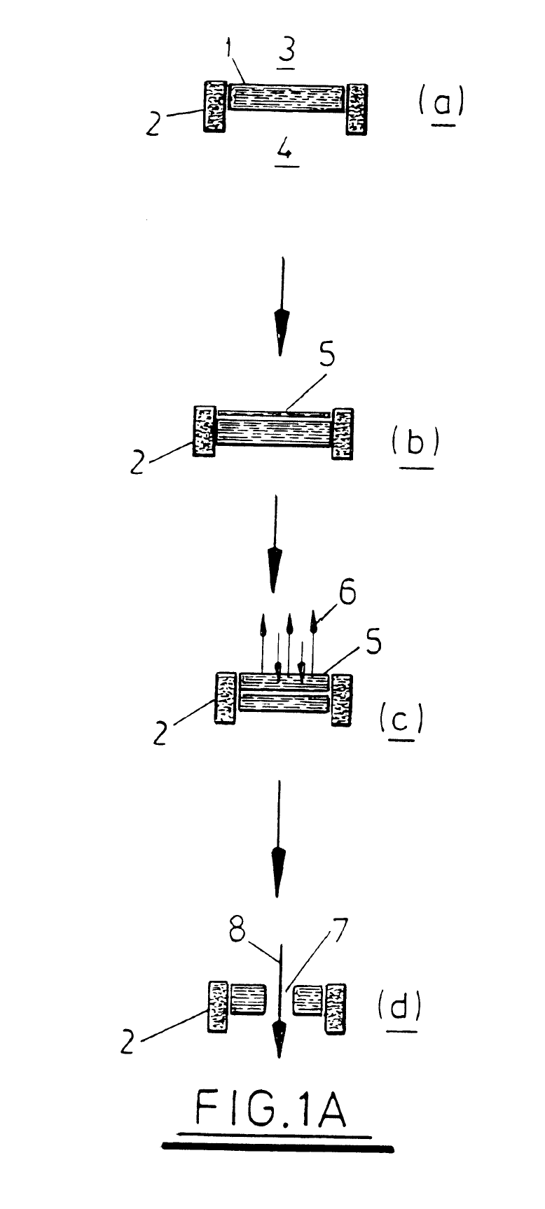

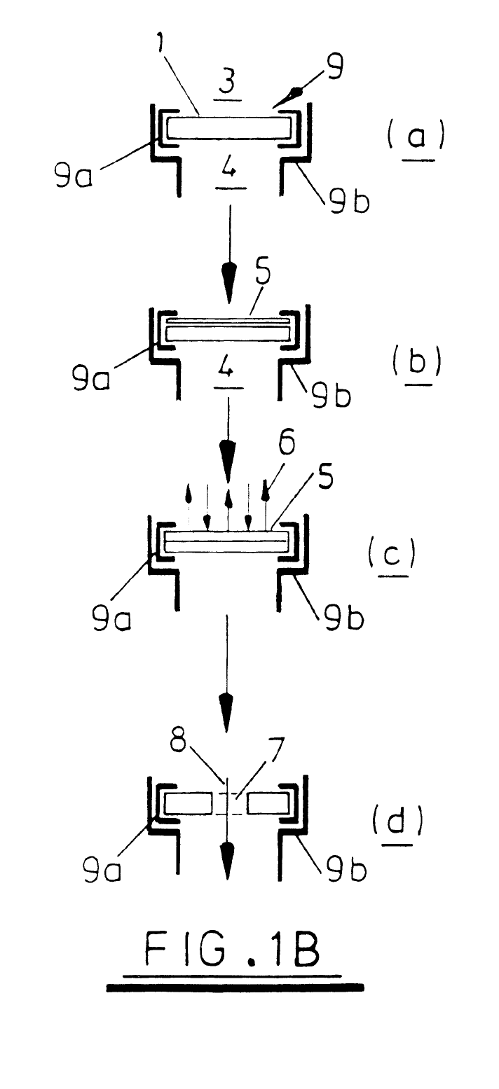

FIG. 1A illustrates the manner in which a barrier in the form of a plug as employed in the invention forms a gel and subsequently dissolves;

FIG. 1B is similar to FIG. 1A but shows the barrier provided in the form of a cartridge;

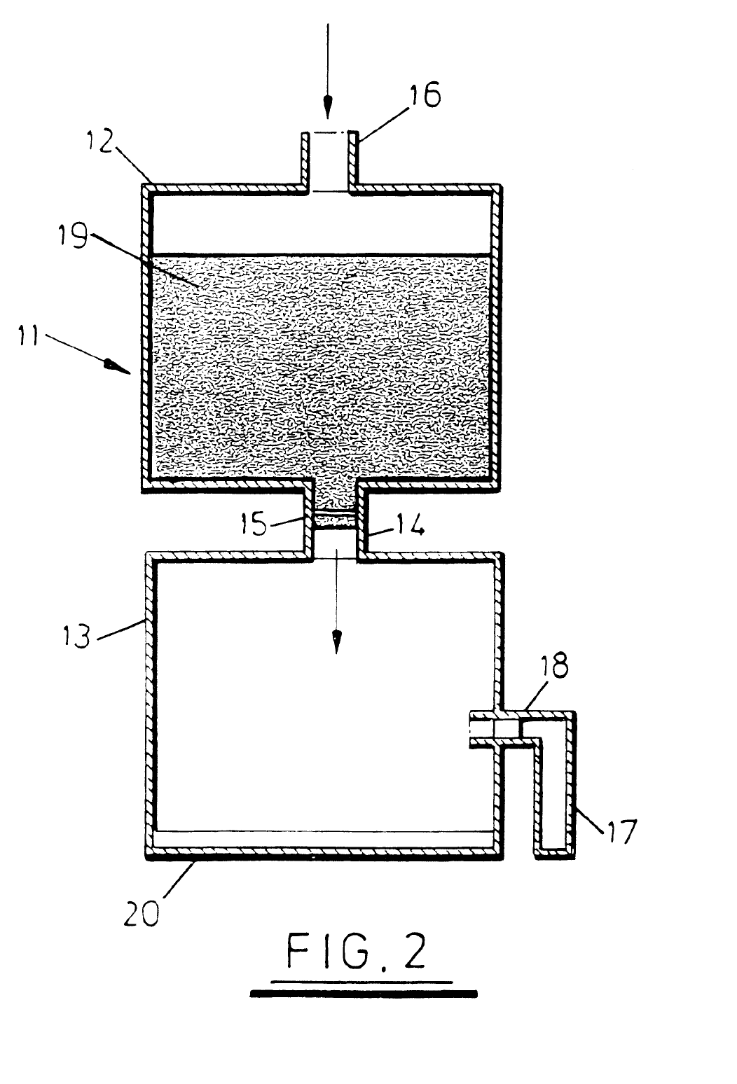

FIG. 2 illustrates a first embodiment of delayed release device in accordance with the invention;

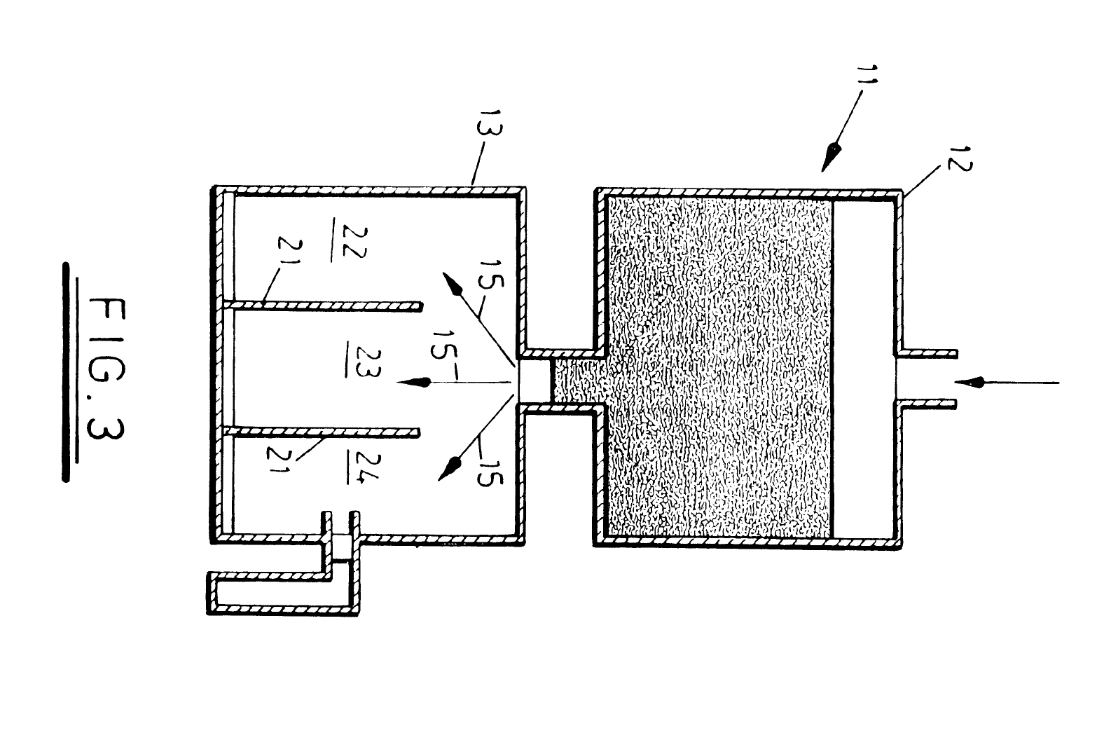

FIG. 3 illustrates a modification of the device shown in FIG. 2;

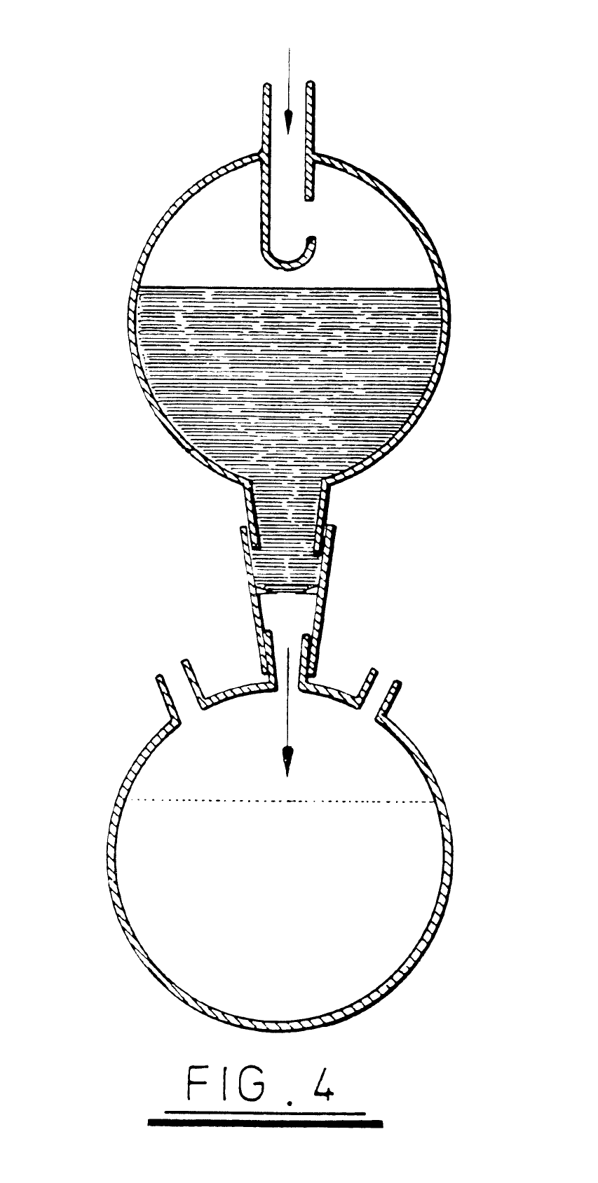

FIG. 4 illustrates a prototype apparatus employed in the procedures of Examples 1 and 2; and

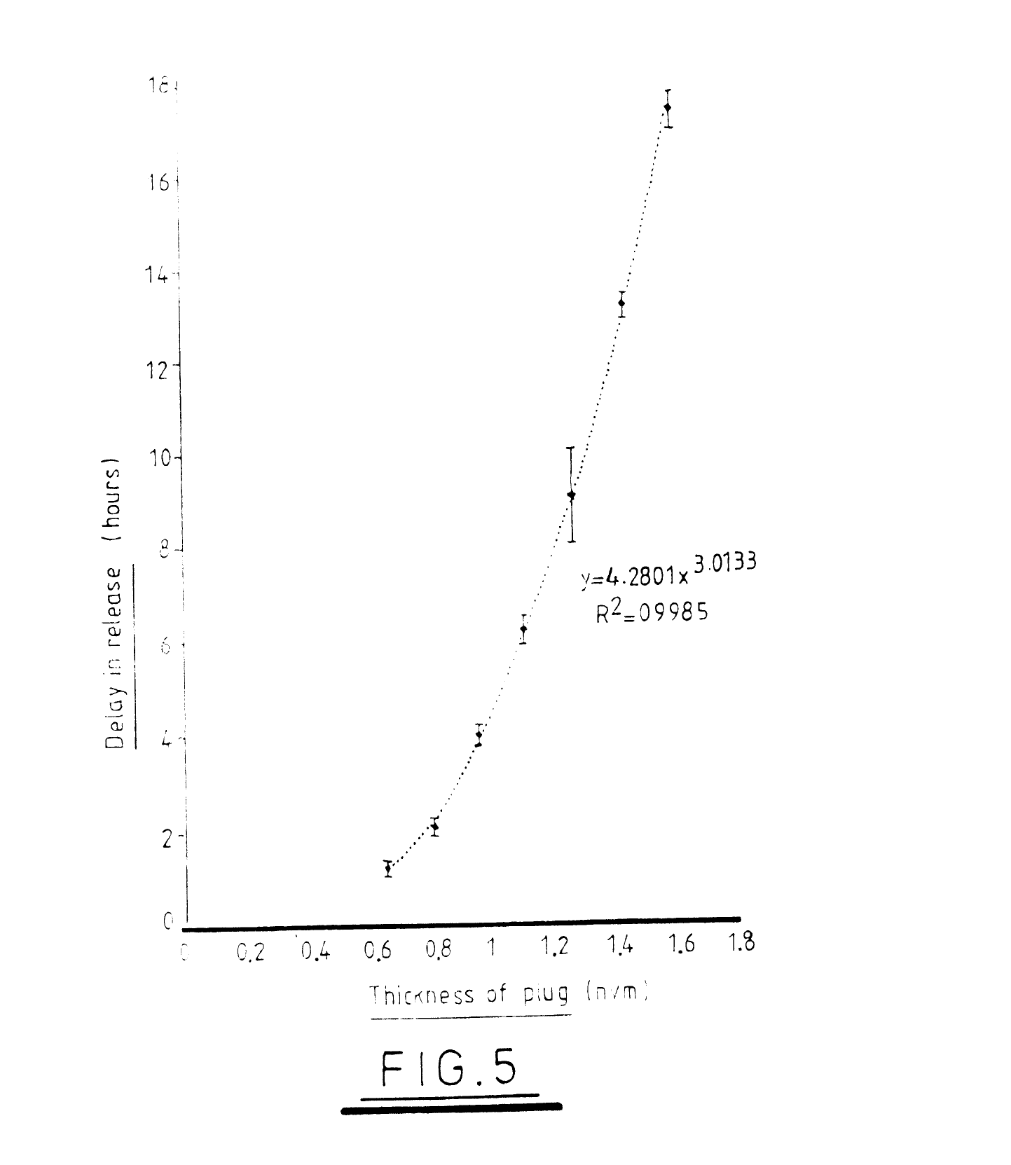

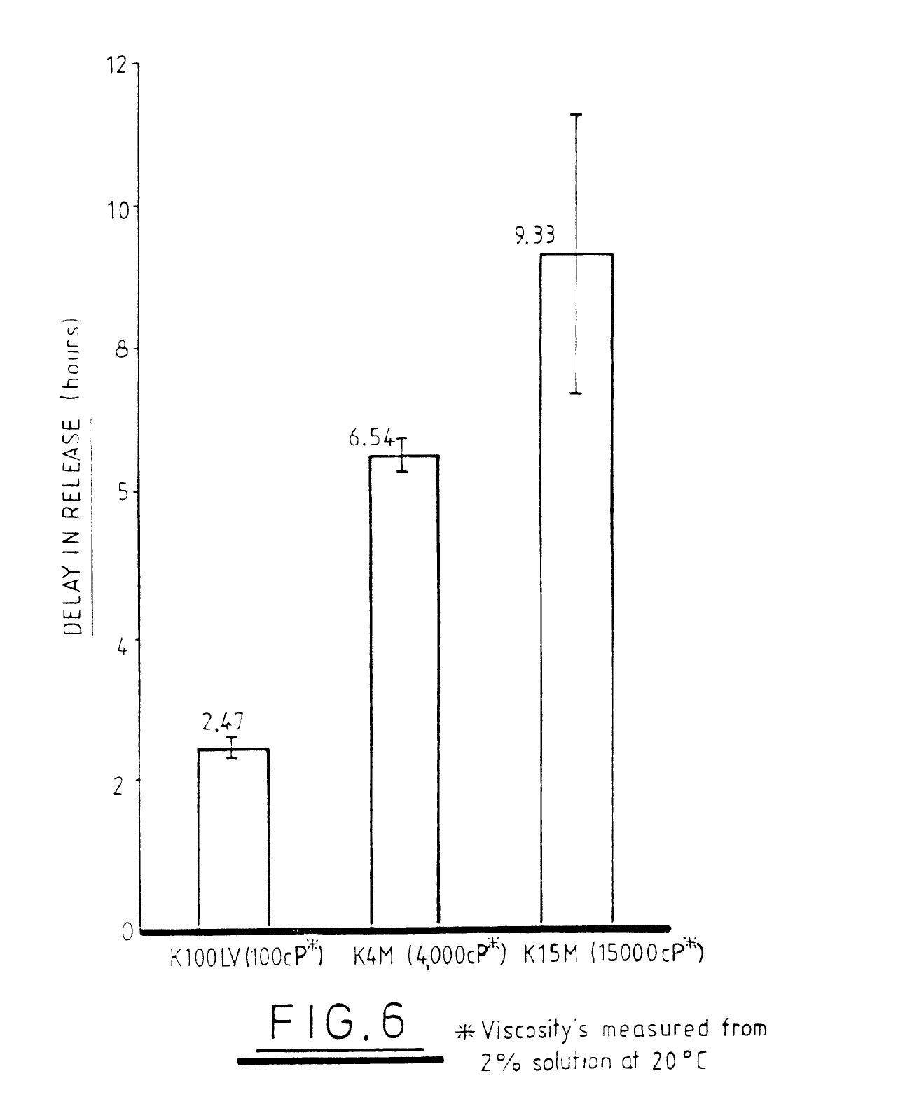

FIGS. 5-6 illustrate the results of Examples 1-3.

FIG. 1A illustrates the manner in which a plug 1 (in the form of a disc) as employed in the present invention hydrates to form a gel and dissolves to allow for mechanical failure of the plug. For the purposes of FIG. 1, it is assumed that the plug 1 is located in position in an annular housing 2 and serves to provide a barrier between a culture of micro-organisms in a liquid medium 3 and a region 4 into which the culture is to be discharged.

On initial wetting of the plug 1 (by the liquid medium 3), polymer molecules at the upper surface of the disc start to hydrate and form a gel layer 5 (see FIG. 1(b)) from which there is disentanglement and dissolution of polymer molecules—as represented by the upwardly pointing arrows 6 (see FIG. 1(c)). As water permeates into the disc, the thickness of the gel layer 5 increases until a maximum thickness is reached at which polymer dissolution and rate of water permeation are equal. Any swelling of the plug 1 results in radial expansion thereof thus ensuring that the plug is initially firmly retained by, and within the ring 2. Hydration and dissolution do however continue and reduce the mechanical strength of the plug 1 until it can no longer withstand the pressure of the liquid 3 resulting in a failure of the plug (depicted as formation of a central breech 7) through which liquid flows under gravity as represented by arrow 8 (see FIG. 1(d)). The aforementioned swelling may serve to delay any “collapse” of the plug by loss of adhesion at and around its peripheral edges.

FIG. 1B is somewhat similar to FIG. 1A and like parts in the two drawings are designated by the same reference numerals. However, in the arrangement of FIG. 1B, the barrier between liquid medium 3 and region 4 is provided by a cartridge 9 comprised of the plug 1 of polymeric barrier material encircled by a peripheral frame 9a whereby the major faces of plug 1 remain exposed. The frame 9a locates on a seat 9b as shown. The manner in which plug 1 remain hydrates, dissolves and fails is as described for FIG. 1A. Subsequent replacement of the cartridge is a simple matter.

Referring to FIG. 2, there is illustrated one embodiment of delayed release device 11 in accordance with the invention for use in the detection of microorganisms. The illustrated device 11 comprises an upper chamber 12 capable of communicating with a lower chamber 13 via a neck 14. Provided in the neck 14 is a plug 15 of a polymeric material capable of forming, in the presence of an aqueous medium, a gel which will dissolve in the medium. The polymeric material may, for example, be METHOCEL K100LV.

Further features of the illustrated apparatus are an inlet 16 for the upper chamber and a transparent side-arm 17 capable of communicating with the lower chamber 13. Provided in the side-arm 17 is a further gellible plug 18 for the purpose described below.

In carrying out a test for detecting a micro-organism, the test sample together with a pre-enrichment medium (as depicted by reference numeral 19) is introduced into the upper chamber 12 and a selective media 20 is provided in chamber 13. The device is then subjected to conditions providing for the recovery of sub-lethally damaged bacteria.

During culture of the medium in chamber 12, the plug 15 begins to gel and to dissolve in the medium 19 (as described on relation to FIG. 1). At a certain point, the plug 15 loses its mechanical integrity with the result that the contents of chamber 12 are discharged into the selective media 20 contained in the lower chamber 13. The amount of culture discharged from chamber 12 is such that the gellible plug 18 (in side-arm 17) is below the upper level of the media now contained in lower chamber 13.

The selective media functions in a manner known per se so that the organisms to be detected (if present) grow to levels facilitating detection.

During culture of the selective medium, plug 18 forms a gel which dissolves into the medium contained in chamber 13.

After a certain period of time, plug 18 loses its mechanical integrity (as described in relation to FIG. 1) so that media from chamber 13 is discharged into the transparent side-arm 17 as an indication that the selective enrichment procedure has proceeded for a pre-determined period of time so that a detection operation may be effected.

The illustrated device may be formed in a number of ways. Thus, for example, the chambers 12 and 13, the neck 14 and inlet 16 may be produced of a relatively rigid plastics material by injection moulding. However in a more preferred implementation of the device, the chambers 12 and 13 have flexible walls and may, in effect, be in the form of bags, e.g. of the type used as “blood bags” or “stomacher bags”. When the chambers 12 and 13 are formed as bags, the device may be used in conjunction with a stomacher which will effect gentle mixing of the media in chambers 12 and 13.

FIG. 3 illustrates a modification of the apparatus shown in FIG. 2 in which the lower chamber 13 is provided with dividers 21 which sub-divide chamber 13 into compartments 22-24. If desired, a different selective enrichment media may be provided in each of these compartments 22-24.

Once the plug 15 is breached, the media from chamber 12 is discharged and, as indicated by the arrows 25, is received in compartments 22-24.

In a further modification (not illustrated) the upper chamber 2 may also be sub-divided into three compartments provided one each above the compartments 22-24 and communicating therewith via respective conduits each containing a gellable plug. In this modification, each compartment of the upper chamber may contain a different sample and/or pre-enrichment medium and each compartment of the lower chamber may contain a different selective media.

The following Examples illustrate, for certain polymers, firstly how it is possible to obtain gellable plugs with different breach times and, secondly, the behaviour of the polymers on exposure to aqueous media.

### EXAMPLE 1

Polymer plugs of METHOCEL K100LV were prepared on a Beckman single punch press using a die and punch of 19 mm diameter. The Methocel was obtained in powder form and pressed under a force of 4 metric tonnes equating to a pressure of 42.7 kg/mm² for a period of 30 seconds. Plugs of different thickness were obtained by use of amounts of the polymer in the range 0.25 to 0.065 g. Dies and punches were not lubricated prior to compression due to the hydrophobic nature of the most widely used lubricants. No adhesion to the dies was encountered. The polymeric discs formed were carefully removed from the die and the thickness measured using Vernia callipers (+/−0.01 mm).

The prototype apparatus illustrated in FIG. 4 was used for determining the time for which various polymer plugs maintained their integrity on exposure to an aqueous medium. The prototype was constructed using Quickfit (RTM) glassware apparatus. A 250 ml anticlimb splashguard adaptor (Aldrich (RTM)) functioned as the top compartment and a 250 ml three neck round bottom flask was used as the lower compartment. The compartments were connected by an expansion adaptor (Quickfit No. XA43). A stainless steel platform of external and internal diameters 19 and 13 mm respectively was cemented into the centre of the adaptor and used to support the polymeric plugs under test.

The metal platform within the adapter was smeared with vacuum grease (Dow Chemicals) to avoid leakage between the plug and platform. Once the plug was positioned and centred on the platform, 250 ml of de-ionised water was slowly poured into the first compartment. This equated to an approximate height of 14 cm above the platform and plug. This equates to an approximate pressure of 1.37 Pa for de-ionised water on the surface of the plug.

The time of release was measured by a change in conductivity in the second vessel created as a result of water entry.

FIG. 5 illustrates the release times obtained using plugs of METHOCEL K100LV of varying thickness. It will be seen from FIG. 4 that a release time of about 1 hour was obtained with a plug thickness of 0.6 mm whereas a release time approaching 18 hours was obtained with a plug thickness of about 1.6 mm.

### EXAMPLE 2

Plugs having a thickness of 1 mm were prepared using the procedure described in Example 1 from hydroxypropyl methyl cellulose polymers (METHOCEL Grade K) of different viscosity. The grades used were K100LV (100 cP), K4M (4000 cP) and K15M (15000 cP), the viscosities being measured for a 2% solution of the polymer in water at 20° c. The plugs were tested using the apparatus shown in FIG. 4 and the results obtained are shown in FIG. 6.

It will be seen from FIG. 6 that Grades K100LV (100 cP), K4M (4000 cP) and K15M (15000 cP) gave release times of 2.47, 6.54 and 9.33 hours respectively.

### EXAMPLE 3

Using 1 gm of polymer, discs were prepared using the procedure described in Example 1 from each of METHOCEL Grades K100LV, K4M and K15M.

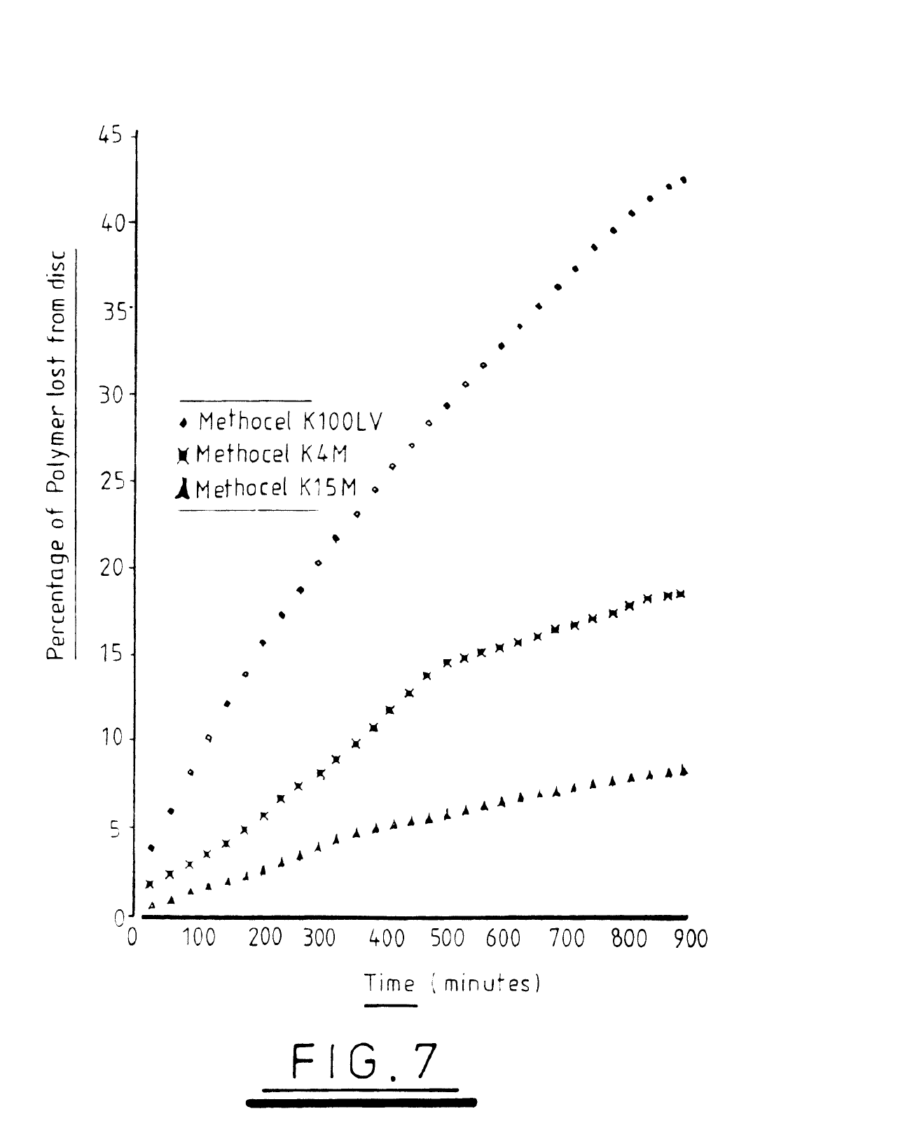

The dissolution properties of the discs were investigated. For this purpose, a needle was driven into the centre of the disc which was then suspended in 250 ml of de-ionised water contained in a glass beaker maintained at 37° C. and covered with aluminium foil. Dissolution media was pumped through a quartz cuvette at a rate of 0.7 l hr⁻¹ and absorbency readings were taken continuously using a Lambda K12 Spectrophotometer. From the absorbency readings in the region 200-300 nm the percentage of polymer lost from the disc was calculated with respect to time. The results are shown in FIG. 7 which clearly demonstrates dissolution of the polymer.

1. A method of treating a culture of microbial organisms in a liquid medium comprising providing the liquid medium (containing the microbial organisms) in a vessel having a discharge aperture provided with a polymeric barrier to liquid flow, effecting the treatment, and utilising liquid in the vessel to cause the barrier to degrade to undergo a loss of mechanical strength or integrity to provide for failure of the barrier and discharge of the liquid medium.

2. A method as claimed in claim 1 wherein the degradation of the barrier is caused by at least partial dissolution thereof in the liquid.

3. A method as claimed in claim 1 wherein the liquid converts the barrier to a gel and the gel at least partially dissolves in the liquid to provide for failure of the barrier.

4. A method as claimed in claim 3 wherein the liquid is an aqueous medium.

5. A method as claimed in claim 4 wherein the polymeric barrier is capable of forming, in the presence of the aqueous medium, a hydrated gel which will dissolve in the medium to provide for failure of the barrier.

6. A method as claimed in claim 5 wherein the polymeric material is a cellulose ether containing methoxyl groups.

7. A method as claimed in claim 6 wherein the cellulose ether contains 15% to 35% substitution by methoxyl groups.

8. A method as claimed in claim 6 or 7, wherein the cellulose ether further contains hydroxypropyloxyl groups.

9. A method as claimed in claim 8, wherein the cellulose ether contains 4% to 15% substitution by hydroxypropyloxy groups.

10. A method as claimed in claim 9 wherein the cellulose ether contains 15% to 35% substitution by methoxyl groups and 4% to 15% substitution by hydroxypropyloxy groups.

11. A method as claimed in claim 10, wherein the cellulose ether contains 19% to 24% methoxyl substitution and 7% to 12% hydroxypropyloxy substitution.

12. A method as claimed in any one of claims 6 to 11, wherein the cellulose ether has a viscosity of 50 to 100,000 cp for a 2% solution.

13. A method as claimed in any one of claims 1 to 12 wherein the treatment effected to the culture is recovery, resuscitation, growth or enrichment.

14. A method as claimed in claim 13 wherein on failure of the barrier the culture is discharged into a selective medium.

15. A method as claimed in claim 13 or 14 wherein provision is made for sustained release of an agent into the culture.

16. A method as claimed in claim 15 wherein the agent for sustained release is provided in a tablet or the like provided in said medium.

17. A method as claimed in claim 15 wherein the agent for sustained release is provided in the barrier layer.

18. A method as claimed in any one of claims 15 to 17 wherein the agent for sustained release is a carbon source to encourage growth.

19. A method as claimed in any one of claim 15 to 17 wherein the agent for sustained release is a selective agent.

20. A method as claimed in any one of claims 1 to 19 wherein, on failure of the barrier, the culture is discharged through the aperture under gravity.

21. A method as claimed in any one of claims 1 to 20 wherein the barrier layer is provided with a layer of a pH responsive polymer.

22. A method as claimed in claim 1 wherein the barrier is of an at least partially crystalline material and the fluid causing degradation of the barrier disrupts the crystallinity to provide for barrier failure.

23. A method as claimed in any one of claims 1 to 22 wherein degradation of the barrier is enhanced by energy provided from externally of the vessel.

24. A method as claimed in claim 23 wherein said energy in a laser beam.

25. A method as claimed in claim 23 wherein said energy is ultrasound.

26. A method of isolating microbial organisms involving the steps of (i) resuscitation or pre-enrichment and (ii) selective enrichment, wherein the step (i) is effected using an aqueous media in a vessel having a liquid discharge aperture provided with a barrier to liquid flow of a polymeric material capable of forming, in the presence of the aqueous medium, a hydrated gel which will dissolve in the medium to provide for failure of the barrier, and on failure of the plug discharging the culture from step (i) into a selective medium.

27. A method as claimed in any one of claims 1 to 26 wherein, at the outset, the polymeric barrier is a tight fit in the discharge aperture.

28. A method as claimed in any one of claims 1 to 27 wherein the polymeric barrier is provided in the form of a replaceable cartridge.

29. Apparatus for use in the method of any one of claims 1 to 28 comprised of a vessel capable of holding a liquid medium and having a discharge aperture provided with a polymeric barrier to liquid release, said barrier being capable of undergoing degradation by the liquid in the vessel to provide for failure of the barrier and discharge of liquid medium wherein the vessel provides a first chamber, the apparatus further comprises a second chamber connected to the first chamber via said discharge aperture in which the polymeric barrier is provided, and the walls of the first and second chambers are of a flexible material.

30. Apparatus as claimed in claim 29, wherein the second chamber is external of the first chamber.

31. Apparatus as claimed in claim 29 to 30, wherein the first chamber is an upper chamber and the second chamber is a lower chamber.

32. Apparatus as claimed in claim 31 wherein the second chamber is sub-divided into a plurality of compartments.

33. Apparatus as claimed in claim 32 wherein the first, upper chamber is subdivided into a plurality of compartments and each such compartment has a respective discharge aperture provided with a barrier of the polymeric material and being positioned one above each of the compartments of the second chamber.

34. Apparatus for use in the method of any one of claims 1 to 28 comprised of a vessel capable of holding a liquid medium and having a discharge aperture provided with a polymeric barrier to liquid release, said barrier being capable of undergoing degradation by the liquid in the vessel to provide for failure of the barrier and discharge of liquid medium wherein the vessel provides a first upper chamber, the apparatus comprises a second lower chamber sub-divided into a plurality of compartments and the first, upper chamber is sub-divided into a plurality of compartments, each such compartment of the first chamber having a respective discharge aperture provided with a barrier of the polymeric material and being positioned one above each of the compartments of the second chamber.

35. Apparatus as claimed in claim 34, wherein the walls of the first and second chambers are of a flexible material.

36. Apparatus as claimed in any one of claims 29 to 35 wherein the polymeric barrier is a tight fit in the discharge aperture.

37. Apparatus as claimed in any one of clam 29 to 36 wherein the polymeric barrier is provided in the form of a replaceable cartridge.

38. A cartridge for use in the apparatus of claim 37, said cartridge comprising the polymeric barrier material and a frame encircling said material.
